SEQUENCE LISTING (9 sequences)

SEQ ID NO: 1 - DNA, 22 bp, Artificial Sequence
  Description of Artificial Sequence Synthetic oligonucleotide
ctgatgagtccgttaggacgaa

SEQ ID NO: 2 - DNA, 22 bp, Artificial Sequence
  Description of Artificial Sequence Synthetic oligonucleotide
ttcgtcctaacggactcatcag

SEQ ID NO: 3 - DNA, 37 bp, Artificial Sequence
  Description of Artificial Sequence Synthetic oligonucleotide
tgctcttctgatgagtccgttaggacgaaactatgga

SEQ ID NO: 4 - DNA, 37 bp, Artificial Sequence
  Description of Artificial Sequence Synthetic oligonucleotide
tccatagtttcgtcctaacggactcatcagaagagca

SEQ ID NO: 5 - DNA, 47 bp, Artificial Sequence
  Description of Artificial Sequence Synthetic oligonucleotide
agctttgctcttctgatgagtccgttaggacgaaactatggagggcc

SEQ ID NO: 6 - DNA, 39 bp, Artificial Sequence
  Description of Artificial Sequence Synthetic oligonucleotide
ctccatagtttcgtcctaacggactcatcagaagagcaa

SEQ ID NO: 7 - DNA, 39 bp, Artificial Sequence
  Description of Artificial Sequence Synthetic oligonucleotide
ntccatagtttcgtcctaacggactcatcagaagagcan

SEQ ID NO: 8 - RNA, 17 nt, Artificial Sequence
  Description of Artificial Sequence Synthetic CEA target sequence
uccauagucaagagcau

SEQ ID NO: 9 - RNA, 38 nt, Artificial Sequence
  Description of Artificial Sequence Synthetic CEA hammerhead ribozyme sequence
augcucuucugaugaguccguuaggacgaaacuaugga

(19) United States
(12) Patent Application Publication
(10) Pub. No.: US 2004/0138158 A1
(43) Pub. Date: Jul. 15, 2004

(54) Cea-expression inhibiting ribozymes and methods for the treatment of cancer based thereon

(76) Inventor: Hartmut Juhl, Hamburg (DE)

Correspondence Address: PILLSBURY WINTHROP, LLP, P.O. BOX 10500, MCLEAN, VA, 22102, US

(21) Appl. No.: 10/469,091

(22) PCT Filed: Feb. 25, 2002
(86) PCT No.: PCT/US02/05257

(51) Int. Cl. A61K 48/00

(52) U.S. Cl. 514/44

(57) ABSTRACT

The present invention provides a method of treatment or prophylaxis of cancer wherein of carcinoembryonic antigen (CEA) plays a role in a subject in need thereof including administering to the subject 1. an effective amount of an agent capable of inhibiting the expression CEA. The preferred agents include ribozymes. The invention also provides a method of potentiating or enhancing the effect of a cancer treatment including in addition to the cancer treatment administering to a subject in need thereof an effective amount of an agent capable of inhibiting the expression of CEA.

### RELATED APPLICATIONS

The present application claims priority to U.S. Provisional Application Serial No. 60/270,585 filed Feb. 23, 2001 the contents of which are hereby incorporated by reference in their entirety.

### BACKGROUND OF THE INVENTION

1. Field of the Invention

The present invention relates to the treatment of cancer, particularly colon cancer. More particularly, the invention relates to the treatment of cancer by inhibition of the expression of the CEA gene.

2. Summary of the Related Art

Colorectal cancer is the third most common malignancy in the United States, with an incidence of 160,000 new patients per year. Only 40% to 50% of the patients survive longer than 5 years (1). Mortality is due to metastatic disease which occurs most often in the liver, followed by the lung. A chance of cure depends on complete surgical removal of the tumor.

5-Fluorouracil (5-FU) is the first line drug for chemotherapy and shows 20-30% response rates in metastatic patients but rarely achieves cure (2).

Numerous clinical studies indicate that carcinoembryonic antigen (CEA) promotes metastatic growth of colon cancer (3). High preoperative CEA serum levels correlate with a poor clinical outcome in colorectal (4), gastric (5), lung (6) and breast cancer (7). Loss of apical CEA expression and diffuse cytoplasmic staining of CEA in colon cancer is also associated with metastatic disease (8) as is CEA expression by circulating colon cancer cells (9). However, although these clinical data strongly suggest a role for CEA in the progression of colon cancer and possibly other malignancies, experimental studies have failed to conclusively determine the biological role of CEA.

CEA was first described as an oncofetal antigen in 1965 (10) and is overexpressed in a majority of carcinomas including cancer of the colon, breast and lung. It is a glycoprotein of approximately 180 kDa, belongs to the immunoglobulin supergene family and is anchored in the cell membrane via a glycosyl phosphatidyl inositol moiety (11).

Overall, the data are conflicting regarding the function of CEA in cancer models. Marked dysregulation of the expression of CEA subgroup members has been noted in colorectal cancer (12) and their differential expression may be important in pathobiochemistry and biology of CEA-positive cancers. Some authors suggest that CEA is a homophilic and heterophilic adhesion molecule (13-16) which may also stimulates release of prometastatic cytokines by Kupffer cells in the liver (17, 18). Other studies propose that CEA serves as a repulsion molecule which increases the mobility of tumor cells (19) but may also function as an immune escape mechanism (20). Because the data from these studies are derived from studying effects of nonphysiologically high levels of CEA, the significance of CEA in metastatic growth has been questioned (22).

To better understand CEA function, it is important to evaluate CEA mediated phenotypic effects within the intact pathophysiological context of cancer cells Thus, there remains a need for the elucidation of the mechanisms through which the CEA plays a role in cancer and the design of therapeutic and diagnosis protocols based on the elucidation of those mechanisms.

### SUMMARY OF THE INVENTION

The present invention provides a method of treatment or prophylaxis of cancer wherein of carcinoembryonic antigen (CEA) plays a role in a subject in need thereof comprising administering to the subject 1. an effective amount of an agent capable of inhibiting the expression CEA. The preferred agents comprise ribozymes, in particular, a ribozyme expressed by an oligonucleotide comprising a nucleic acid sequence selected from the group consisting of TGCTCTT; ACTATGGA; TCCATAGT; AAGAGCA; CTGATGAGTCCGTTAGGACGAA; TTCGTCCTAACGGACTCATCAG; TGCTCTTCTGATGAGTCCGTTAGGACGAAACTATGGA; TCCATAGTTTCGTCCTAACGGACTCATCAGAAGAGCA; 5′-agcttTGCTCTTCTGATGAGTCCGTTAGGACGAAACTATGGAgggcc-3′; and 5′-cTCCATAGTTTCGTCCTAACGGACTCATCAGAAGAGCAa-3′.

The method of the invention is particularly suitable for the treatment of cancers selected from the group consisting of colon; breast; lung; cervical; prostate; and head and neck cancer, and more particularly, colon cancer.

The invention also provides a method of potentiating or enhancing the effect of a cancer treatment comprising in addition to said cancer treatment administering to a subject in need thereof an effective amount of an agent capable of inhibiting the expression of carcinoembryonic antigen (CEA). Treatment that can be effectively potentiated or enhanced according to the invention include chemotherapy, radiation, and/or antisense therapy.

### BRIEF DESCRIPTION OF THE DRAWINGS

FIG. 1: Characterization of tet-off system and ribozymes in vitro.

(A) Luciferase assay to determine tetracycline dependent regulation of gene expression in stably transfected HT29 colon cancer cell clones. HT29 cells expressing the tetracycline Transactivator (tTA) protein were transiently tranfected with the pUHC 13-3 plasmid DNA that codes for luciferase under the control of the tet-O binding site. Luciferase activity was measured 36 hours after transient transfection in the absence (−tet) and presence of 1:g/ml tetracycline (+tet).

(B) In vitro cleavage assay to determine the activity of CEA-targeted ribozyme Rz4 (sequence shown in the inset). A CEA transcript of 814 nt was coincubated with 100-fold excess of Rz4 for various time intervals (right 5 lanes). The expected cleavage products of 421 nt and 393 nt length became visible after 0.5 hour. As a control (left) served CEA RNA without additon of Rz4 (M=molecular weight marker).

FIG. 2: CEA-targeted ribozyme cleavage activity in vivo (HT29/Rz4 colon cancer cells).

(A) FACS-analysis to determine CEA expression of HT29/Rz4 cells and the corresponding Northern Blot (inset). Ribozyme expression was inhibited by adding 1:g/ml tetracycline (+tet). The inset shows the corresponding Northern Blot: 1=MC38 murine colon cancer cell line overexpressed with human cDNA of CEA, 2=MC38 cells (CEA negative), 3=ribozyme expressing HT29/Rz4 (−tet, CEA level diminished), 4=ribozyme inhibited HT29/Rz4 (+tet, CEA level normalized). The lower bands represent the 18s RNA loading control.

(B) Western Blot analysis to determine the 24 hour time kinetic of CEA reconstitution after inhibition of ribozyme expression by 1:g/ml tetracycline (left panel). The right panel shows as a control the CEA expression of HT29/Rz4 cells at the starting and endpoint of the experiment without tetracycline (−tet).

FIG. 3: (A+B) shows the cDNA microarray analysis of CEA depleted HT29/Rz4 cells (−tetracycline) and cells which were treated for 24 hours with 1:g/ml tetracycline (+tetracycline) with respect to cell cycle and apoptotic genes (cells were harvested from culture flask when confluent, compare right panel of (D). (C+D) gives data of the corresponding phenotype.

(A) Expression of cell cycle genes (n=36) of HT29 colon cancer cells with depleted (−tetracycline) and restored CEA levels (+tetracycline). Eight genes were higher expressed when CEA levels were restored while 13 genes were upregulated when ribozymes inhibited CEA. The x-fold increase of gene expression is added to the gene names in parenthesis. A 1.5-fold change of gene expression was regarded as significant (hatched line).

(B) Expression of apoptotic genes (n=29) of HT29 colon cancer cells with depleted (−tetracycline) and restored CEA levels (+tetracycline). While only one gene was upregulated when CEA levels were restored, 9 genes were higher expressed when ribozymes inhibited CEA (p<0.05). The x-fold increase of gene expression is added to the gene names in parenthesis. The hatched line represents a 1.5-fold change of gene expression.

(C) Distribution of cell cycle subpopulations at low and high CEA levels (−tet and +tet, respectively) is shown in the left panel. The right panel illustrates the proliferation rate of HT29/Rz4 cells with low and high CEA levels (−tet and +tet, respectively). Additionally, the data from HT29/tTA control cells are demonstrated.

(D) Analysis of the apoptotic rate of HT29/Rz4 cells with depleted and restored CEA levels (−tet and +tet, respectively). The left panel shows the results when cells were harvested at a semiconfluent stage (>70% single cells) and gives the number of stained cells for, first, AnnexinV, second, AnnexinV+Propidiumiodide (PI), third, only PI and, finally, the combination of AnnexinV and AnnexinV+PI stained cells. The last one represents both early and late apoptotic cells. The right panel demonstrates the data from confluent grown cells at the time of harvesting (corresponding to cDNA microarray, (B)).

“*”=p<0.05, “***” p<0.0001.

FIG. 4: AnnexinV-Propidiumiodide (PI) staining to determine the apoptotic rate in CEA depleted HT29/Rz4 cells and cells in which ribozyme expression was shut off by tetracycline 24 hours before analysis. Cells were harvested at a semiconfluent stage to assure no influence of dense cell growth on apoptosis (compare FIG. 3D).

(A) shows the FACS analysis of HT29/Rz4 cells without (−tet) and with tetracycline (+tet) which were treated for 48 hours with 25 U/ml (−interferon. The lower panel illustrates the number of apoptotic cells (AnnexinV and AnnexinV+PI stained cells) and its change under (−interferon treatment. The 2.5 fold increase of the apoptotic rate in −tet cells was statistically highly significant (p<0.0001). 2×10⁴ cells were counted in this experiment.

(B) shows the FACS analysis of HT29/Rz4 cells without (-tet) and with tetracycline (+tet) which were treated for 48 hours with 50:M 5-FU. The lower panel illustrates the number of apoptotic cells (combination of AnnexinV and AnnexinV+PI staining) and its change under 5-FU treatment. The 2.8 fold increase of the apoptotic rate in −tet cells was statistically highly significant (p<0.0001). In this experiment 1×10⁵ cells were counted.

FIG. 5: In vitro aggregation assay to determine CEA dependent aggregate formation. HT29/Rz4 and HT29/Rz4-2 were treated with and without 1:g/ml tetracycline (+/−tet) to modify CEA levels. HT29/tTA-5 cells served as a negative control cell line. 24 hours after tetracycline was added, cells were seeded in soft agar and the number of cell clusters (>80 μm diameter which consist of at least 10 cells) were determined (p<0.05). “*” indicates p<0.05.

FIG. 6: Illustrates the results from a colony formation assay. 2 weeks after seeding of confluently grown the HT29/Rz4, HT29/Rz4-2 and HT29/tTA-5 (control) cells into soft agar with and without 1:g/ml tetracycline (+/−tet), colonies larger than 80:m were counted using an image analyzer. “*” indicates p<0.05.

FIG. 7: (A) Quantification of CEA dependent HT29/Rz4 tumor cell seeding in the lung of nude mice. Tumor sections of 5 different levels of the lung were immunostained for human cytokeratin (left) and the numbers of cells were counted (right). Shown are the mean values 1 hour and 24 hours after tumor cell tail vein injection (n=5 per group).

(B) Determination of CEA dependent metastatic growth in vivo. HT29/Rz4 cells were injected in the tail vein of two groups of nude mice (n=5). One group obtained doxycycline enriched food to block CEA-targeted ribozyme expression (+dox) while the second group received doxycycline-free food (−dox). After 6 weeks microscopic slides of the lungs were immunostained for human epithelial cells to identify (left) and quantify (right) metastatic lesion of >50 cancer cells. Overall, one mice in the “−dox” group (Ribozyme on CEA down) developed only one metastatic lesion while all 5 mice of the “+dox” group (Ribozyme off =normal CEA) had several metastatic lesions. “**” indicates p=0.001.

### DETAILED DESCRIPTION OF THE PREFERRED EMBODIMENTS

1. Material and Methods

Generation of ribozyme expressing HT29 cell lines. The generation of CEA specific ribozymes and the characterization of tetracycline controlled ribozyme expression, cell clones HT29 Rz4 and HT29/Rz4-2 which were derived from human HT29 colon cancer cells as described below.

Generation of Constructs.

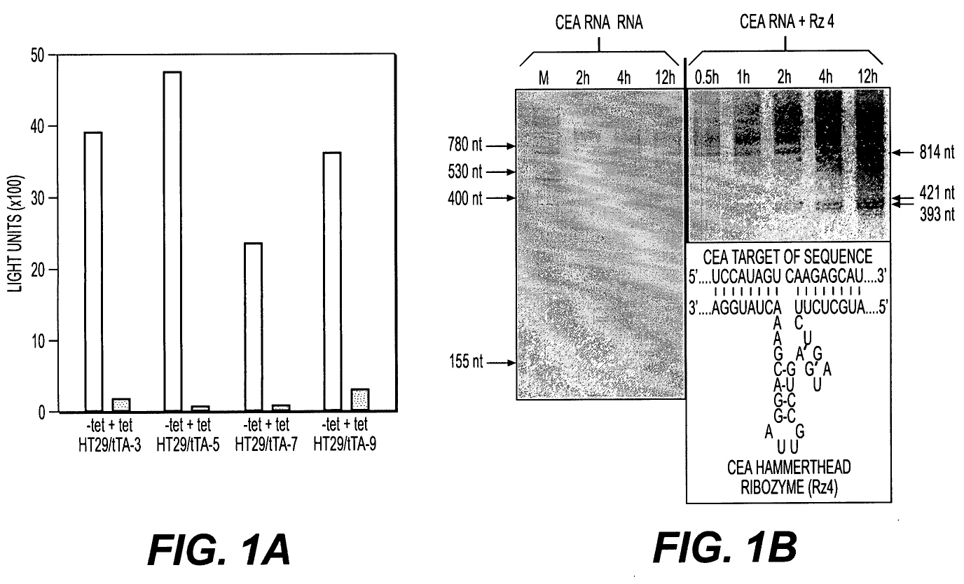

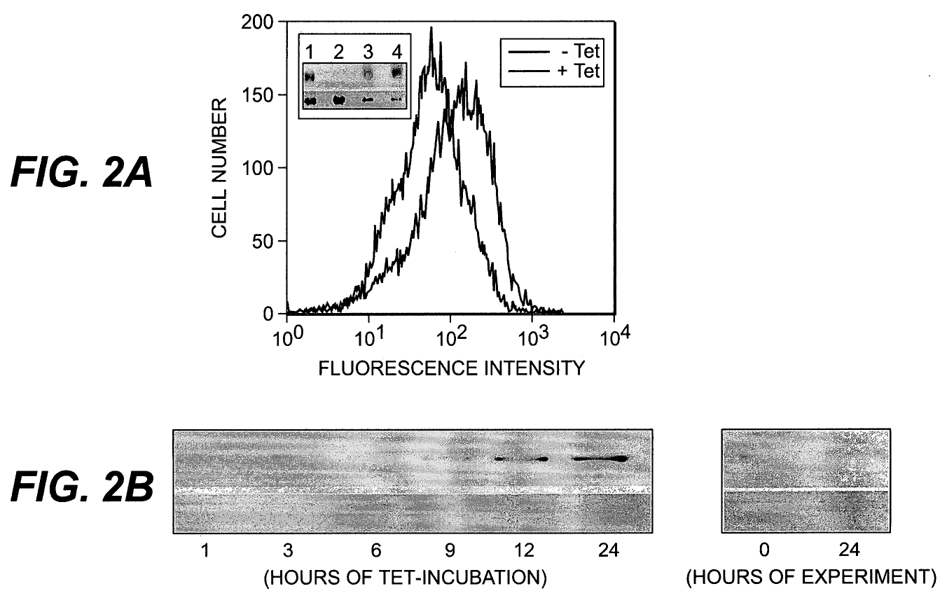

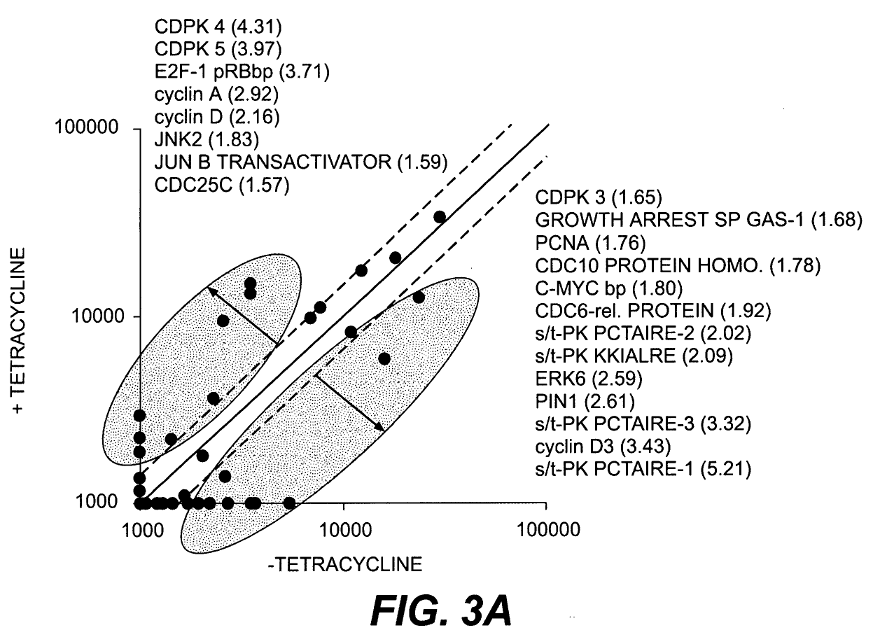

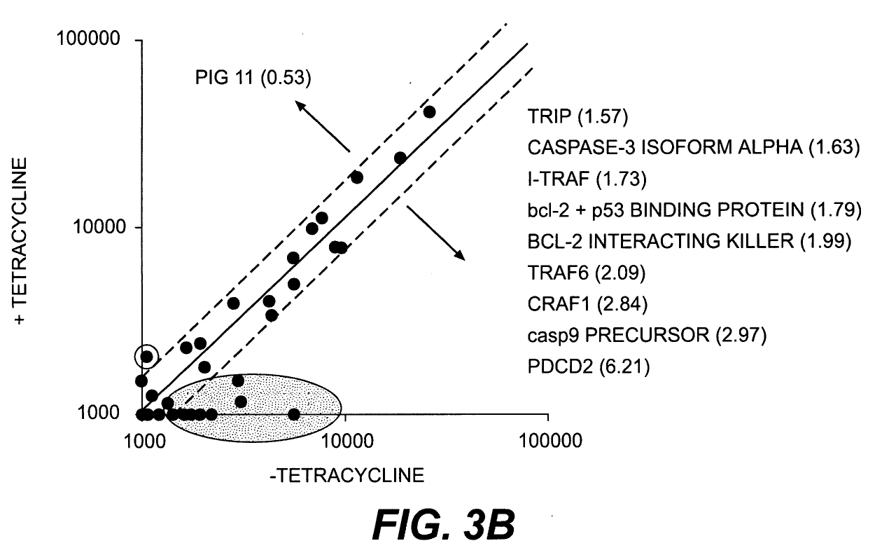

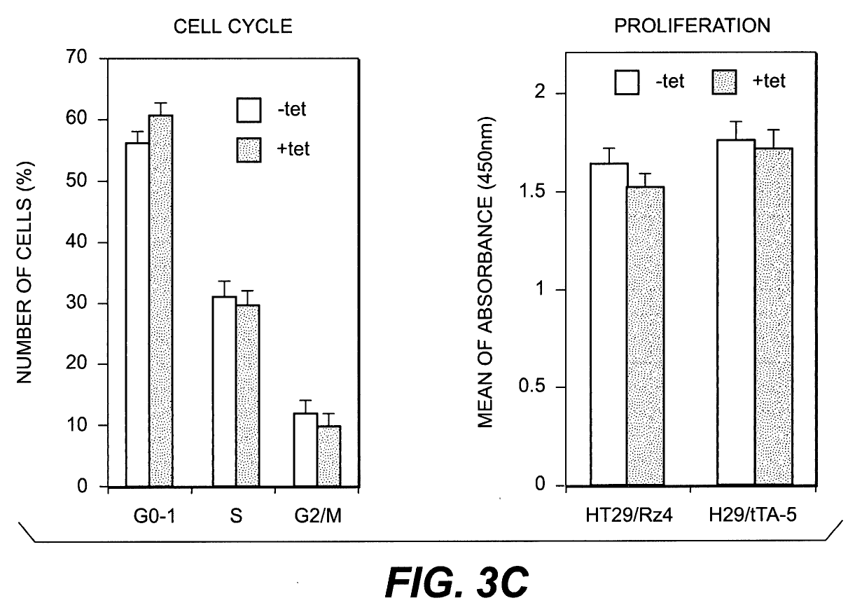

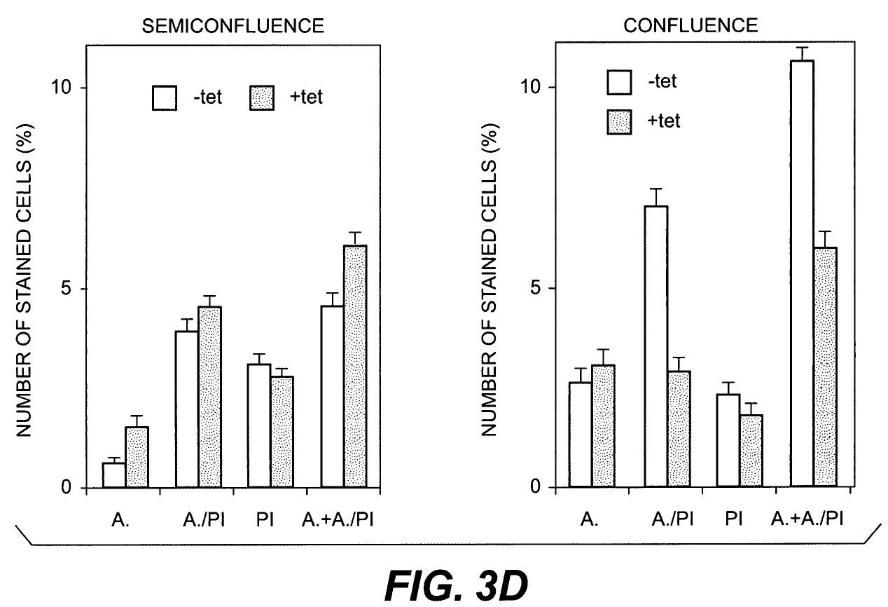

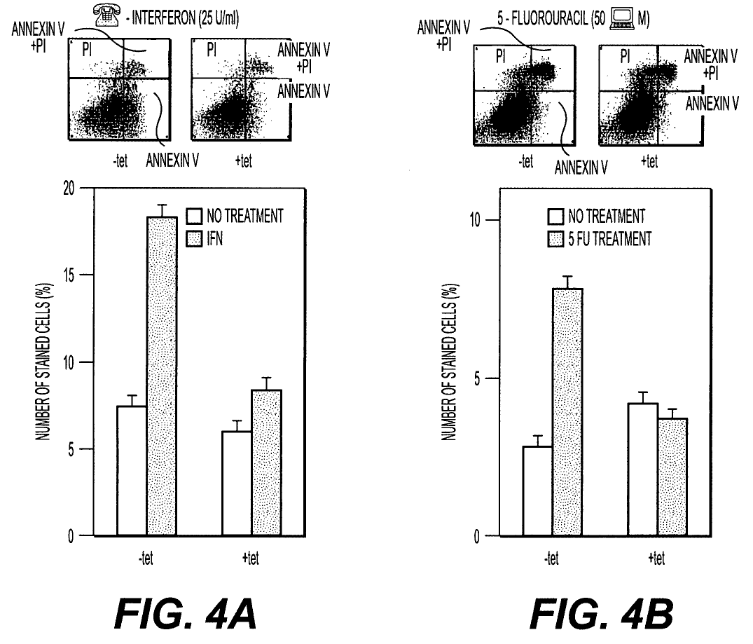

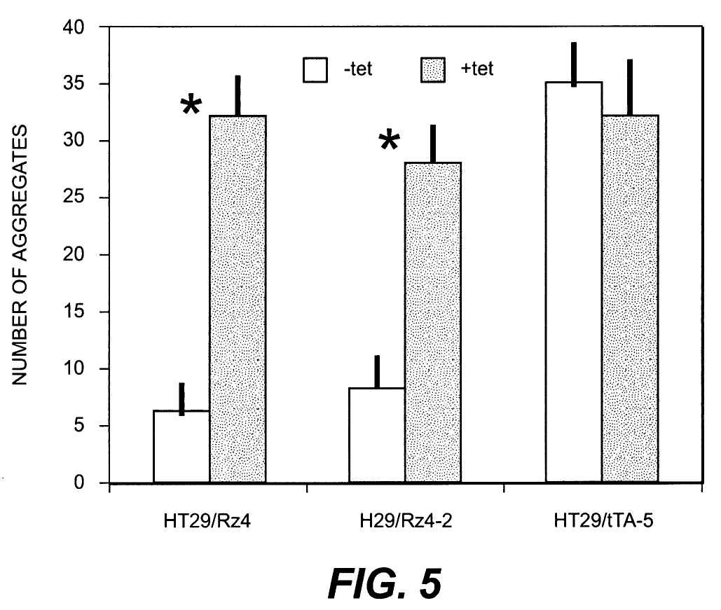

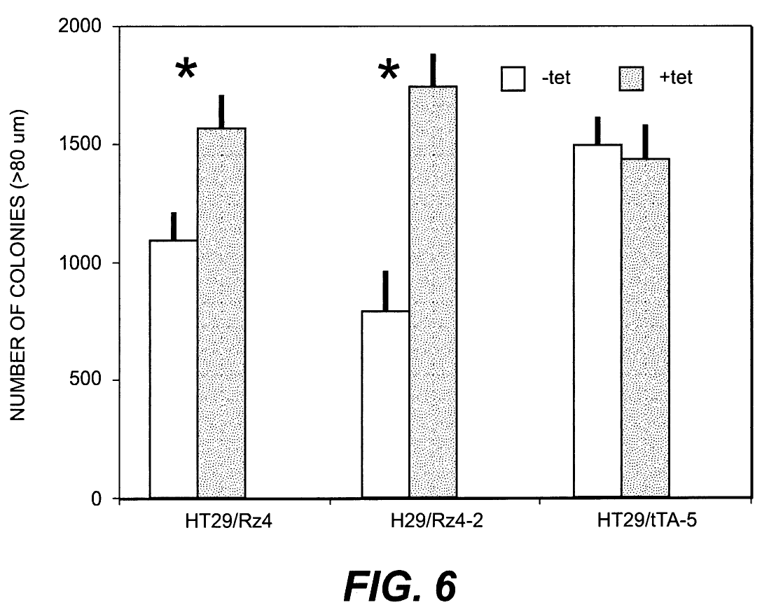

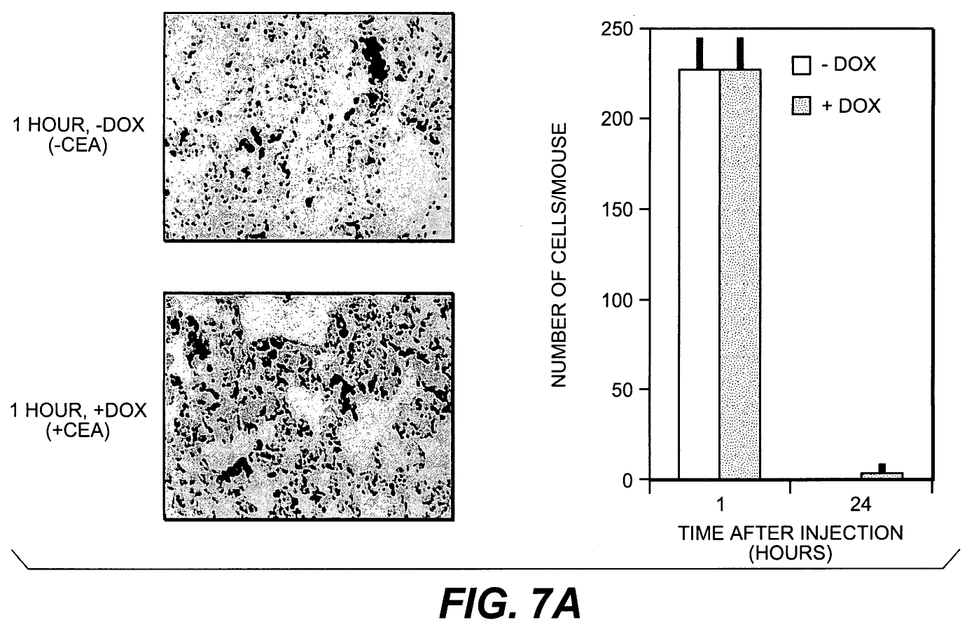

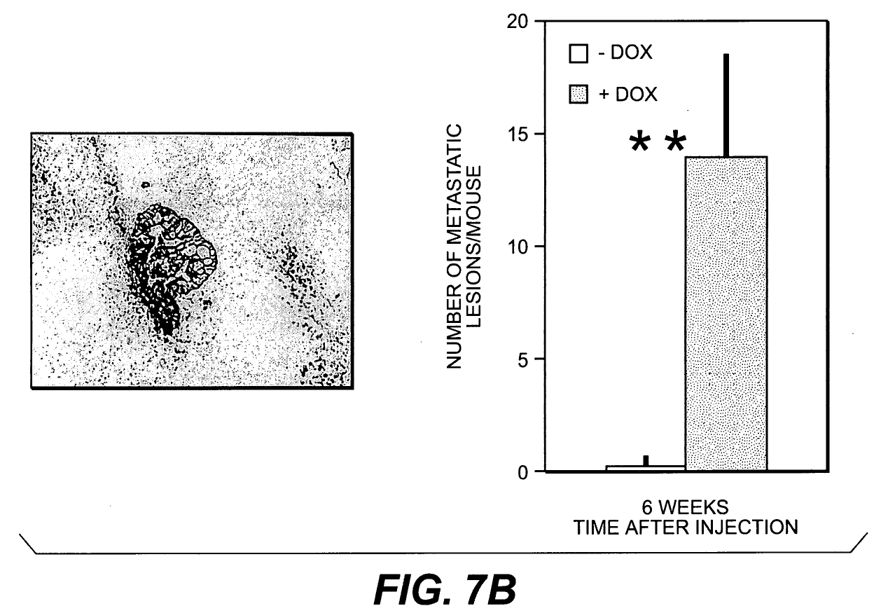

Plasmids expressing the tetracycline transactivating (tTA)/VP 16 fusion protein (pUHG15-1) and the tTA/heptameric operator binding site (tet-O; pUHC13-3) (23) were obtained from Dr. Bujard (Heidelberg, Germany). The ribozyme expression plasmid (pTET) was derived from pUHC13-3 and modified as described (24). The CEA-targeted hammerhead ribozyme expression vector was prepared as described (25). Blast search of the Rz-sequence confirmed the specificity for CEA mRNA. The following ribozyme coding sense and antisense oligonucleotides were annealed and ligated into the HindIII- and NotI-restriction site of pTET: 5′-agcttTGCTCTTCTGATGAGTCCGTTAGGACGAAACTATGGAgggcc-3′ (sense) and 5′-cTCCATAGTTTCGTCCTAACGGACTCATCAGAAGAGCAa-3′ (antisense) with lower case letters indicating HindIII-/NotI restriction site overhangs, bold capital letters showing CEA specific antisense regions and italic capital letters indicating the hammerhead ribozyme core sequence. The resulting ribozyme expression plasmid pTET/Rz2113 contains CEA specific antisense flanking regions of 7 nucleotides (nt) on 5′ and 8 nt on 3′ ends of the 22 nt catalytic hammerhead ribozyme core sequence (FIG. 1B, insert), that target it to the B3 domain of CEA. Additionally, the ribozyme DNA was ligated into the pRc/CMV vector (Invitrogen, San Diego, Calif.) which allows performance of an in vitro cleavage assay.

In Vitro Cleavage Assay.

To generate smaller in vitro transcripts the full length CEA sequence which was ligated into the pBluescript II KS (+/−) vector (Invitrogen) was cut with NotI and PSTI (New England BioLabs, Beverly, Mass.). This yielded a 765 bp CEA fragment containing the recognition sequence for Rz2113 which was religated in a pBluescript SK (+/−) vector and linearized by Not 1. ApaI (Life Technologies, Gaithersburg, Md.) was used to linearize the pRc/CMVRz2113 vector. The enzymes were heat inactivated and the transcripts were refined using a Chroma SPIN-30+DEPC-H₂O column (Clontech, Palo Alto, Calif.). A run-off transcription reaction for the ribozyme and target RNA was carried out with T7 RNA polymerase using a MAXIscript Transcription Kit (Ambion, Austin, Tex.). After DNA digestion (DNase I treatment) transcripts were refined with the Chroma columns. The purified RNA products were combined (100 fold molar excess of ribozyme transcript) and resuspended in a 50 μl reaction volume containing 50 mM Tris-Cl (pH 7.5) and 1 mM EDTA and heated 3 min at 95° C. As a negative control the same amount of CEA RNA was incubated under the same conditions without the ribozyme.

The cleavage reaction was performed as described (26). Aliquots (10:1) were removed after 0.5, 1, 2, 4, and 12 hours and the reaction was stopped by the addition of Ambion Loading buffer II including 40 mM EDTA and stored at −80° C. Samples were boiled briefly and separated on a 6% TBE Urea/polyacrylamide gel (Novex, San Diego, Calif.). Products were visualized by silver staining (Novex) according the manufacturers protocol with the exception that 2 mg/l Na₂S₂O₃ was added to the developing reaction to reduce background staining.

Cell Lines and Transfections.

Human HT29 colon cancer cells were obtained from American Type Culture Collection (ATCC, Rockville, Md.) and were maintained in continuous culture at 37° C./5% CO₂ using IMEM (Life Technologies Inc., Gaithersburg, Md.) supplemented with glutamine and 10% heat-inactivated fetal bovine serum (FBS). Murine MC38 colon cancer cells and human CEA expressing MC38 cells were kindly provided by Dr. J. Shively, Beckman Research Institute, Duarte, Calif. MC38 cells were stable transfected by electroporation using an eukaryotic expression vector (neomycin resistance gene) which contained the full length cDNA of human CEA. CEA expressing clones were obtained after G418 selection. CEA expression levels exceeded the CEA expression of HT29 cells by a factor of 2 as determined by FACS analysis (data not shown).

HT29 cells were transfected using LipofectAmine (Life Technologies). Briefly, cells at 50-70% confluency were incubated for 5 hours with plasmid DNA mixed with LipofectAmine (7 μl LipofectAmine/1 μg plasmid DNA) in serum-free Opti-MEM medium (Life Technologies) at 37° C. in 5% CO₂. The transfection medium was then replaced with normal growth medium and 36 hours later supplemented with the respective drugs for selection of stable integrants. HT29 stably expressing tetracycline regulated CEA targeted ribozymes were generated in a two-step transfection protocol. In a first step, HT29 cells were transfected with 10 μg of pUHG15-1 plasmid DNA and 1 μg of pRc/CMV plasmid DNA (Invitrogen) to provide Geneticin (G418, Life Technologies) resistance. After selection for stable integrants in the presence of G418 at 0.7 mg/ml, individual tTA expressing clones were isolated. To test the clones for tTA expression and tetracycline regulation, the cells were transiently transfected with pUHC 13-3 plasmid DNA that contains a luciferase cDNA under the control of the tet-O binding site and cultured in the absence and presence of 1 μg/ml tetracycline (Sigma), respectively. Cell lysates were prepared 36 hours after transfection and luciferase activities were measured in a luminometer as described (24).

Clone HT29/tTA-5 demonstrating the best tetracycline regulation of luciferase activity was used for further transfections with the ribozyme expression plasmids. HT29/tTA-5 cells were then transfected with 10 μg of pTET/Rz2113 mixed with 1 μg of pZeo (Invitrogen) to provide Zeocin resistance. Clones, obtained after selection with 0.4 mg/ml Zeocin and 1 μg/ml tetracycline, were screened for tetracycline regulated CEA expression using FACS analysis. We established a clonal HT29 cell line (HT29/Rz4) in which CEA was up- and downregulated by approximately 50%.

Northern Analysis.

Total cellular RNA was isolated with the RNA STAT-60 method (Tel-Test, Friedenswood, Tex.), and 30 μg were separated and blotted as described (27). A ³²P labeled CEA cDNA probe (541 nt PstI fragment) was hybridized, washed and exposed to film for 16 hours. To correct for variability in loading 18s RNA bands were used or blots were stripped and reprobed with a Glyceraldehyde-3 phosphate dehydrogenase (GAPDH) cDNA probe (Clontech). Relative band intensities were measured by densitometry.

Fluorescence Activated Cell Sorting (FA CS).

Cells were detached using 0.02% EDTA in PBS, washed with ice cold PBS containing 7.5 mM sodium azide and 5×10⁵ cells were incubated with 2 μg of anti-CEA antibody (Cymbus Biotechnologies LTD, Chandlers Ford, Hants, UK) for one hour at 4° C. Incubation was stopped by two washes with PBS and cells were incubated for an additional 30 min with Fluorescein (DTAF)-conjugated Goat Anti-Mouse IgG+IgM antibody (1:100, Jackson ImmunoResearch, West Grove, Pa.) under cold and dark conditions.

After two final washings, cells were resuspended (300 μl PBS) and fixed by the addition of 100 μl 4% paraformaldehyde. The mean values of fluorescence intensity of 10,000 cells were determined by FACS (FACStar plus; BectonDickinson). Unlabeled cells and cells labeled with secondary antibody alone served as negative controls.

Western Blot Analysis.

Cells were lysed in buffer containing 50 mM Tris-HCl pH 8.0, 150 mM NaCl, mM β-glycerophosphate disodium salt, 0.05% deoxycholic acid sodium salt, 1% NP-40, 50 mM sodium fluoride, 20 mM sodium pyrophosphate, 1 mM EGTA, 1 mM sodium orthovanadate, and protease inhibitors (2:g/ml Leupeptin, 2:g/ml Aprotinin, 1:g/ml PepstatinA, and 100:g/ml Pefabloc).

Cell lysates were assayed for total protein content, equal amounts of total protein (40:g) were loaded into pre-cast 4-20% gradient Tris-glycine polyacrylamide gels (Fisher Scientific, Pittsburgh, Pa.) and gels were run at 130V in buffer containing 25 mM Tris, 192 mM glycine and 0.1% (w/v) SDS, pH 8.3 (Bio-Rad Laboratories, Hercules, Calif.).

Gels were transferred onto Immobilon-P nylon membranes (Millipore, Bedford, Mass.) for 3 hrs at 150 mA per gel, the membranes were dryed overnight, rehydrated, and blocked for 1 hr in PBST (0.05% Tween20) and 5% nonfat dry milk. Membranes were probed with a 1:500 diluted monoclonal mouse antibody to CEA (Cymbus Biotechnologies) followed by incubation with 1:5000 diluted rabbit anti-mouse IgG antibody conjugated to horseradish peroxidase (Jackson ImmunoResearch). Immunoreactive bands were visualized by enhanced chemiluminescence (Amersham Pharmacia Biotech, Piscataway, N.J.). cDNA microarray. For cDNA microarray analysis, the “Atlas™ Human Cancer cDNA Expression Array” (Clontech) was used which covers 588 cancer related genes, arranged in 13 functional groups (cell cycle/growth regulators, intermediate filament markers, apoptosis, oncogenes/tumor suppressors, DNA damage response/repair and recombination, cell fate and development, receptors, cell adhesion and motility, angiogenesis, invasion regulators, cell-cell interactions, Rho family and small GTPases, growth factors and cytokines). The microarray analysis was performed according to the manufactures guidelines. HT29/Rz4 cells were cultivated in culture medium. 24 hours before harvesting equivalent amounts of cells were distributed in 6 culture flask and 1 :g/ml tetracycline was added in 3 flask to block ribozyme expression. Cells were detached by 0.02% EDTA/PBS and cells with and without tetracycline treatment were pooled. The cells in all flask were at comparable levels of confluence at the time of harvesting. HT29/tTA5 cells were treated in the same way and served as a control.

RNA was extracted according to the manufacturers protocol followed by DNAse I treatment. We performed a Northern Blot for CEA which confirmed intact RNA and demonstrated a 50% downregulation of CEA mRNA before the experiment were continued.

For cDNA synthesis we used 50:g total RNA which was converted to ³²P labelled first strand cDNA by means of SuperScriptII reverse transcriptase (Life Technologies). Unincorporated nucleotides were removed by CHROMA SPIN-200 column chromatography (Clontech). The first two fractions with highest activity were pooled. Equivalent amounts of cpm were used to minimize loading differences. After prehybridization of the membrane for 30 min at 68° C. in ExpressHyb (Clontech) supplemented with 200:g/ml salmon sperm DNA (Life Technologies), the heat denaturated probe was added. Hybridization was performed over night and after washing the membranes were first exposed to an X-ray film followed by phophoimager analysis.

Cell Cycle Analysis.

For cell cycle analysis we used the Vindelov staining method as described (28). Tetracycline treated (1:g/ml) and untreated cells were harvested, 2×10⁶ cells were resuspended in 100:1 of 40 mM citrate/DMSO buffer. After addition of trypsin inhibitor and Ribonuclease A (10 min), staining was done using propidiumiodide and cell cycle analysis was performed in a flow cytometer.

Aggregation Assay.

To determine aggregate formation we modified a soft agar assay described previously (25). Single cell suspensions of tumor cells were prepared drawing cells through a 30G×½″ needle. Cells were kept in suspension for 20 min to allow aggregation and were seeded into liquid agar at 42° C. When the soft agar solidified an image analyzer was used to determine the number of aggregates larger than 80:m in diameter which corresponds to a cell colony of at least 10 cells.

Proliferation Assay.

Cells were plated on microtiter plates (5×10³/well) and cultivated using culture medium with and without 1:g/ml tetracycline, respectively. After 6 days WST-1 reagent was added (Boehringer Mannheim, Germany) and incubated for 30 min. The absorbance were determined at 450 nm using an ELISA microreader.

Determination of Apoptosis.

Cells (1×10⁶) were harvested, washed twice with 500:1 cold PBS, pH 7.4 and resuspended in 100:1 of propidium iodide-annexinV-FITC dual staining solution as described (TACS™ AnnexinV-FITC protocol; Trevigen, Gaithersburg, Md.) and incubated in the dark for 15 min at room temperature. 400:11× binding buffer was added to cell suspension and cells were analyzed by flow cytometry within 1 hour.

Colony Formation Assay.

To determine whether CEA's antiapoptotic function has an effect on in vitro tumor growth we used a soft agar colony formation assay which was described previously (25). Confluently grown tumor cells were harvested, pooled and divided into different tubes of which one contained 1:g/ml of tetracycline to block ribozyme expression. Single cell suspension of 1×10⁴ cells were stirred in 0.35% Bactoagar (Life Technologies) and layered on top of 1 ml of a solidified 0.6% agar layer in a 35-mm petridish. The agar was solved in IMEM culture media supplemented with 10% FCS and, as indicated, contained 1:g/ml tetracycline. After 14 days at 37° C. and 5% CO₂, colonies larger than 80 μm in diameter were counted using an image analyzer.

Tumor Growth in Nude Mice.

5×10⁶ tumor cells without and with (24 hours) 1:g/ml doxycycline (a tetracycline analogue which is absorbed by the intestine) were injected into the tail vein. A group of mice received food containing 200 mg/kg doxycyline (Bioserve, Frenchtown, N.J.) to continuously block ribozyme expression. Groups of mice (n=5) were sacrificed after 1 hour, 24 hours and 6 weeks, respectively. The lungs were snap frozen in liquid nitrogen and cryostate sections were prepared at 5 different levels of the lung for immunohistochemistry.

Immunohistochemistry.

Immunostaining with Kl-1 anti-human cytokeratin antibody (Imunotech, Marseille, France) which does not crossreact with murine cells was used to identify tumor cells following a previously published protocol (35). The numbers of tumor cells and metastatic lesions, respectively, were determined using light microscopy.

In Vitro Experiments

Generation of tTA-Expressing HT29 Cells.

Various HT29/tTA clones were transiently transfected with pUHC13-3 plasmid DNA, coding for luciferase, and several clones were derived which showed high luciferase activity (FIG. 1A). Optimal regulation was obtained in HT29/tTA-5 cells showing a 100-fold difference of luciferase activity which was inhibited to background levels by tetracycline. CEA expression of HT29/tTA-5 cells and HT29 wildtype cells were compared by FACS analysis and no differences were detectable with respect to CEA expression (data not shown).

Efficacy of CEA-Targeted Ribozymes in Vitro and in HT29 Cells.

Ribozyme activity was first tested in an in vitro cleavage assay and demonstrated a complete digestion of the CEA transcript into the expected 421 nt and 393 nt RNA cleavage products within 12 hours (FIG. 1B).

HT29/tTA-5 cells were transfected with the pTET/Rz2113 plasmid and we identified several clones by FACS analysis in which CEA was downregulated in a tetracycline dependent manner including the HT29/Rz4 clone. This clone showed a consistent 50% downregulation of CEA and was selected for subsequent experiments (FIG. 2A). The reduction of CEA protein by 50% was confirmed by a Western Blot. In accordance to these data, we also found a 50% reduction of CEA mRNA using Northern Blot analysis (FIG. 2A, inset, lanes 3 and 4). As a negative control for the Northern Blot we used CEA-nonexpressing MC38 murine colon cancer cells (lane 2 of inset). MC38 cells stably transfected with a CEA expression vector were used as a positive control (lane 1 of inset).

To determine the time kinetics of ribozyme activity with respect to CEA translation we performed a Western Blot and measured the CEA protein level in HT29/Rz4 cells at various time intervals after the addition of tetracycline. FIG. 2B illustrates the results of this experiment. The blockade of ribozyme expression by tetracycline restored 50% of total CEA within 9 to 12 hours (left panel). Maximal CEA levels appeared after 12 to 24 hours. As a control, CEA levels were determined in HT29/Rz4 cells continuously cultured without tetracycline which showed consistently low CEA levels (right panel).

Analysis of CEA-Dependent Gene Expression.

To identify genes which are potentially affected by CEA, we studied the gene expression profile of HT29/Rz4 cells using the “Atlas™ Human Cancer cDNA Expression Array”. The mRNA levels of HT29/Rz4 cells were analyzed comparing ribozyme-expressing cells (low CEA) and cells which were treated by tetracycline to block ribozyme-expression (normal CEA). To exclude a potential influence of tetracycline we analyzed HT29/tTA-5 cells untreated and treated with tetracycline. Reliable signals (signal intensity >1000) were available for 273 out of 588 genes. We regarded a shift of gene expression by a factor of 1.5 as significant (29). This was the case in 134 genes affecting virtually all gene groups (data available upon request). In our study we focussed on the relation of cell cycle/proliferation and apoptosis because an imbalance of these two pathways is known to affect tumor growth. Ribozyme inhibition (increased CEA levels) induced a change of cell cycle gene expression in a bidirectional and balanced fashion (FIG. 3A). In contrast, elevation of CEA levels significantly shifted apoptotic genes in one direction (p<0.05, Dixon and Mood test) and 9 out of 10 apoptotic genes were downregulated (FIG. 3B). Tetracycline did not affect the expression of these genes as determined in HT29/tTA-5 control cells (data not shown).

To correlate the microarray data with a cellular function we further analyzed cell/cycle and proliferation as well as the apoptotic rate with respect to ribozyme controlled CEA levels.

Analysis of Cell Cycle and Proliferation.

Analysis of cell cycle and proliferation was determined 24 hours after the addition of tetracycline to inhibit ribozyme expression as CEA levels normalized within 12-24 hours after the ribozyme was inactivated by tetracycline (FIG. 2B). Neither the cell cycle nor the proliferation rate of HT29/Rz4 cells were affected by CEA (FIG. 3C).

Analysis of Apoptosis.

Initially, we analyzed the apoptotic rate in relation to the density of cells in culture to determine the impact of cell-cell contact. In accordance to our microarray experiment, we compared the apoptotic rate 24 hours after tetracycline treatment in confluent cells. Additionally, we analyzed semiconfluent cells (>70% appeared in culture as single cells). Interestingly, the level of CEA influenced the apoptotic rate in two directions. When cells were grown to a semiconfluent stage (FIG. 3D, left panel), CEA expressing cells had a slightly but significant higher apoptotic rate than cells with 50% reduced CEA levels (p<0.05). However, when cells became confluent (FIG. 3D, right panel), the apoptotic rate in CEA depleted cells increased 2.5 fold while cells with normal CEA levels were not affected (p<0.0001).

Next, we studied whether the protective effect of CEA was restricted to dense growth conditions (confluence). We treated semiconfluent cell cultures of HT29/Rz4 cells (+/−tetracycline) with various apoptotic stimuli including, UV-light, (−interferon and 5-FU (30, 31).

In an initial experiment, we tested if CEA has a protective function using 200 Joule UV light to induce apoptosis. Under this extreme conditions, HT29 cells with normal CEA levels (+tetracycline) had a significantly reduced apoptotic rate by 30% compared to CEA depleted tumor cells (p<0.05) (data not shown). As shown in FIG. 4A, application of 25 U/ml of (−interferon increased the apoptotic rate 2.5-fold in CEA downregulated cells but had no significant impact on the apoptotic rate of cells with restored CEA levels (p<0.0001).

Finally, 5-FU (50:M) had a similar effect and also demonstrated a significant protective function of CEA (FIG. 4B). A 50% ribozyme-depletion of CEA increased the apoptotic rate 2.8-fold while normal CEA levels completely prevented HT29 cells from undergoing apoptosis (p<0.0001).

There was no effect of 1:g/ml tetracycline on the apoptotic rate of HT29/tTA-5 control cells, in which 25 U/ml of (-interferon and 50:M of 5-FU did not significantly affect the apoptotic rate (data not shown).

The aim of this study was to elucidate potential functions of the carcinoembryonic antigen (CEA) in the biology of colon cancer cells. To analyze the role of CEA we designed specific CEA-targeted hammerhead ribozymes expressed under the control of the tet-off system. This approach has three major advantages compared to previously used methods: first, using ribozymes enables a highly specific knockout of the target molecule (32), second, using the tet-off promoter system allows regulation of CEA levels within cancer cell clones, and, finally, it enables a comprehensive analysis of CEA mediated effects within an intact pathophysiological cellular context.

To comprehensively screen for CEA mediated molecular effects we performed cDNA microarray analysis of HT29 colon cancer cells 24 hours after ribozyme expression was shut off by the addition of tetracycline. Using the “Atlas™ Human Cancer cDNA Expression Array” from Clontech which covers 13 cancer related gene groups, 273 genes generated reliable signals in all arrays and were evaluated for the effect of CEA on their expression level.

Using a 1.5 fold change in gene expression as a cut off (29) approximately half of the genes changed their expression level when CEA was modified (data available upon request), a finding which deserves further extensive analysis. While a shift of expression of individual genes does not predict the cellular phenotype, a dysregulation of genes within functional groups implies phenotypic changes, in particular when a significant shift occurs in an unidirectional manner. In our study we focussed on the relation of cell cycle/proliferation and apoptosis because the balance of these two pathways significantly affects tumor growth and both functional gene groups were significantly affected by ribozyme mediated modification of CEA levels. However, the change of gene expression differed in both groups: while cell cycle genes shifted bidirectionally at elevated CEA levels in a balanced manner, apoptotic genes were unidirectionally downregulated in CEA expressing cells (p<0.05). None of the observed changes were seen in the tetracycline treated HT29/tTA-5 control cell line. In addition, ribozymes lack cleavage activity if there is a mismatch of 2 or more nucleotides (32) and the use of a highly specific target sequence which is unique for human CEA strongly underlines that the observed changes are ribozyme related and CEA specific.

In accordance to our microarray data, we found a significant change of the apoptotic rate but the cell cycle and proliferation rate did not differ between CEA depleted and CEA expressing cells.

However, the apoptosis regulating function of CEA is complex. Recently, it was described that CEA may regulate apoptosis in an indirect way. Ordonez et al. found that CEA overexpression protects cells from anoikis possibly by CEA mediated tumor cell aggregation (33). Our data suggest a direct role of CEA in the regulation of apoptosis which depends on external factors including proximity to other CEA expressing cells. Dense cell growth resulted in a significantly 2.5-fold higher apoptotic rate in CEA depleted cells while semiconfluent conditions resulted in slightly lower apoptotic rate. However, further experiments demonstrated that the protective function of CEA is not restricted to cell proximity. We treated semiconfluently grown cells with various inducers of apoptosis including UV-light, (−interferon and, finally, the colon cancer drug 5-FU. All these treatments demonstrated a protective role of CEA. Taking into account that the CEA level was only reduced by 50% our findings are even more striking and have clinical implication.

Under conditions of external stress (confluent growth, UV-light, (−interferon, 5-FU) CEA serves as a stabilizing factor and protects from apoptosis. This CEA effect is unrelated to its potential adhesive function as described by Ordonez et al. (33) because the protective function was observed under semiconfluent conditions when the cells were attached and equally distributed in the culture flask as single cells. Because no significant differences were seen in cell cycle analysis and proliferation rate we propose that CEA moderates the physiological balance between proliferation and apoptosis. CEA expressing colon cancer cells may have a growth advantage in vivo because the protective function of CEA can help colon cancer cells to survive the hostile conditions they are exposed to during progression. This assumption is supported by in vivo data showing a significantly lower metastatic rate of CEA-depleted HT29/Rz4 cells in nude mice (unpublished data). Furthermore, CEA may also interfere with anti-cancer agents such as 5-FU by inhibiting activation of the apoptotic cascade.

In Vivo Experiments

Efficacy of CEA-targeted ribozymes in HT29 cells. The effect of tetracycline dependent CEA regulation in HT29/Rz4 and HT29/Rz4-2 cells has been discussed in conjunction with the above in vitro data which showed a 50% downregulation of CEA mRNA (Northern Blot) and protein levels (FACS, Western Blot) in ribozyme expressing cells compared to tetracycline treated cells (inhibition of ribozyme expression).

Tumor cell aggregation. We compared the aggregate formation of HT29/Rz4 and HT29/Rz4-2 cells at high and reduced CEA levels (with and without tetracycline treatment). Downregulation of CEA significantly decreased the number of aggregates by 70% (p<0.05). Tetracycline itself did not modify aggregate formation as determined in HT29/tTA-5 control cells which were treated in the same manner (FIG. 5).

Analysis of proliferation and apoptosis. As described in conjunction with the in vitro data discussed above, we found in HT29/Rz4 and HT29/Rz4-2 cells no differences regarding proliferation within 72 hours. However, confluent growth as well as other apoptotic stimuli such as treatment with 5-Fluorouracil and (−interferon, increased the apoptotic rate exclusively in CEA reduced HT29 cells but did not affect cells with normal (baseline) CEA levels.

Colony formation. We used a soft agar colony formation assay to determine the growth rate of HT29/Rz4 and HT29/Rz4-2 cells in relation to CEA levels over a period of 2 weeks. As shown in FIG. 6 we found that cells treated with tetracycline (Rz off=CEA at high baseline level) develop 30 to 50% higher colony numbers compared to cells without tetracycline treatment (Rz on=reduced CEA levels).

CEA dependent tumor cell seeding and growth in vivo. Tumor cells pretreated and untreated with doxycycline were injected in nude mice obtaining doxycycline enriched or free food, respectively. Groups of mice (n=5) were sacrificed after one hour, 24 hours and 6 weeks and lung microsections were analyzed by anti-human cytokeratin immunostaining to quantify the number of tumor cells and metastatic lesions, respectively.

One hour after cell injection we found 230±20 tumor cells/mouse (5 slides each) in mice receiving doxcycline (Rz off=CEA high) in contrast to 235±29 tumor cells/mouse in the untreated group (Rz on=CEA low) (p>0.05). After 24 hours, doxycycline treated mice showed 2+2 tumor cells/mouse and 0 tumor cells/mouse in the untreated group (p>0.05) (FIG. 7A). After 6 weeks all five mice of the doxycycline treated group showed metastatic lung lesions (14.5±4.6 lesions/mouse) while only one of five mice in the untreated group had one metastatic lesion (0.2±0.2 lesions/mouse) (p<0.001) (FIG. 7B). In control experiments HT29/tTA-5 cells, irrespective of doxycycline treatment induced the same numbers of metastatic lesions compared to doxycycline treated HT29/Rz4 cells (data not shown).

CEA expression of tumors in doxycycline treated and untreated mice were not compared because only one small lesion appeared in the “−dox” group. CEA-immunostaining confirmed strong CEA expression in all metastatic lesions of the “+dox” group (data not shown).

The role of CEA in malignant (and normal) conditions is elusive and several functions have been suggested. C. P. Stanner's lab suggested CEA as an intercellular adhesion molecule (3). However, the lab of P.-L. Lollini performed similar studies and found the opposite result using a human rhabdomyosarcoma cell line (36). These conflicting data suggest that the function of CEA depends in part on its interaction with other membrane molecules and may be tissue type dependent. Furthermore, other authors have questioned if the adhesive function of CEA plays any role under physiological and in vivo conditions because an adhesive CEA function has only been clearly demonstrated in vitro in cells overexpressing the gene (22).

CEA has also been implicated as a heterophilic intercellular binding molecule that mediates the colonization of liver and lung by colon cancer cells. For example, in colon cancer cells high CEA expression correlates with the rate of metastatic spread, and hepatic colonization seems to rely on an interaction of tumor cells and Kupffer cells (18, 36). Further studies suggest that CEA might increase the metastatic capability of cancer cells by inducing paracrine effects on normal cells. It has been found that binding of CEA to Kupffer cells induces the release of I1-6, TNF-alpha, I1-1α and I1-1β (17) by binding to hnRNP M4, a recently described receptor of CEA ((37). In turn, these cytokines may enhance the metastatic rate in vivo by downregulating an immune response against tumor cells and/or modify the adhesion molecule expression pattern of endothelial cells in a manner that improved colon cancer cell binding (17, 33). Thus there could be a crosstalk towards endothelial cells.

As discussed above in vitro data disclosed herein has shown an anti-apoptotic function of endogenous CEA in human colon cancer cells. Dense cell growth and treatment with various inducers of apoptosis including UV-light, (-interferon and 5-FU resulted in a significantly 2.5-fold higher apoptotic rate in CEA reduced cells. It hypothesized that under conditions of external stress (confluent growth, UV-light, (−interferon, 5-FU) CEA serves as a stabilizing factor and protects tumor cells from apoptosis. As discussed above, these data were derived by using a tetracycline regulated ribozyme expression model. This approach allows highly specific reduction of CEA because ribozymes lose cleavage activity by a mismatch of only two nucleotides (32). Blast search did not reveal a matching mRNA sequence except for CEA. Therefore, it is reasonable to link the phenotypic effects directly to CEA, although cleavage of a currently unkown mRNA can not completely be excluded.

In contrast to other studies our cell model allows studying the phenotypic effect of endogenous CEA and, thus, study the impact of physiological CEA levels in cancer cells.

In this study we compared the impact of CEA on tumor cell aggregation and protection against apoptosis in vitro with corresponding findings in vivo. So far, an adhesive function of CEA has exclusively been observed in cells which express CEA at high density or have been transfected with a CEA expression vector but was not demonstrated in human colon cancer cells which show normal or low CEA levels such as HT29 cells. Ribozyme mediated reduction of CEA levels by 50% resulted in a 70% decrease in cellular aggregate formation of HT29 colon cancer cells. The addition of tetracycline (which itself did not affect aggregation) completely reversed the ribozyme effect. Considering the data from Landuzzi et al. (36), who did not find an adhesive function of CEA in rhabdomyosarcoma cells, we propose that the adhesive function of CEA is tissue dependent and that the intercellular binding function depends not only on the CEA density but the interaction between other molecules.

Comparing the apoptotic and proliferation rate of high and low CEA expressing HT29 cells we found that confluently grown cells differ regarding their apoptotic rate by approx. 50%. When we grew these cells in soft agar and counted the number of colonies after 2 weeks the imbalance of proliferation and apoptosis resulted in a significantly higher number of colony formation in cells with high baseline CEA levels.

To compare the impact of CEA-dependent aggregation and apoptosis we analyzed the metastatic growth of HT29 cells in nude mice. We assume that differences in aggregate formation alter primarily the phase of tumor cell seeding which occurs within the first hours following tumor cell injection. Interestingly, one hour after tail vein injection of HT29 tumor cells with high and low CEA levels, we found almost identical numbers of single cells and tumor cell aggregates in the lung. 24 hours later virtually all cells were eliminated in both groups. This finding strongly suggests that differences in aggregate formation as seen in vitro had no significant impact on tumor cell seeding in vivo. However, 6 weeks after tumor cell injection, we detected in all mice, which were treated with doxycycline to continuously block ribozyme expression, numerous lung metastases while cells with ribozyme reduced CEA levels did not develop metastatic lesions in 4 out of five mice (p<0.05). These data strongly suggest that CEA has a major effect when tumor cells have already seeded in their metastatic target organ. Animal models have shown that human cancer cells find optimal growth conditions when placed orthotopically in their organ of origin but show a higher apoptotic rate when placed as metastatic lesions (38).

Together with our in vitro data, i.e. increased apoptotic rate under treatment with apoptotic stimuli, the determination that reduced colony formation of CEA diminished HT29 colon cancer cells, suggest that CEA's survival function is a major factor in CEA mediated colon cancer progression. However, other potential functions such as the induction of growth modulating cytokines from endothelial cells (17) may also contribute to CEA's prometastatic role. Our cell model will allow us to elucidate the growth regulating role of CEA in vivo.

In summary, the results presented in this application demonstrate a multifunctional role of CEA in colon cancer cells such as tumor cell aggregate formation and protection against apoptosis. The animal experiments suggests that the growth regulating effect of CEA is of importance for metastatic growth while aggregate formation plays a less significant role in tumor progression.

While the invention has been described in terms of preferred embodiments, the skilled artisan will appreciate that various modifications, substitutions, omissions and changes may be made without departing from the spirit thereof. Accordingly, it is intended that the scope of the present invention be limited solely by the scope of the claims provided below, including equivalents thereof

### REFERENCES

1. Mettlin, C. Recent developments in colorectal cancer epidemiology and early detection. Curr. Opinion Oncol., 2:731-737, 1990.

2. Isacoff, W. H., Borud K. H. Chemotherapy for the treatment of patients with metastatic colorectal cancer. World J. Surg. 21:748-762, 1997.

3. Jessup, J. M., and Thomas, P. CEA and metastasis: a facilitator of site-specific metastasis. In: Stanners, C. P. (ed.) Cell adhesion and comunication mediated by the CEA family, pp.195-222. Amsterdam: Harwood Academic Publishers, 1998.

4. Kos, J., Nielsen, H.-J., Krasovec, M., Christensen, I., Cimerman, N., Stephens, R., Brunner, N. Prognostic values of cathepsin B and carcinoembryonic antigen in sera of patients with colorectal cancer. Clin, Cancer Res., 4:1511-1516, 1998.

5. Nakane, Y., Okamura, S., Akehira, K., Boku, T., Okusa, T., Tanaka, K., Hioki, K. Correlation of preoperative carcinoembryonic antigen levels and prognosis of gastric cancer patients. Cancer, 73:2703-2708, 1994.

6. Vinolas, N., Molina, R., Galan, M. C., Casas, F., Callejas, M., Filella, X., Grau, J., Ballesta, A., Estape, J. Tumor markers in response monitoring and prognosis of non small cell lung cancer: preliminary report. Anticancer Res., 18:631-634, 1998.

7. Ebeling, F., Schmitt, U., Untch Michael. Nagel D., Fateh-Moghadam A., Stieber P., Seidel D. Tumor markers CEA and CA 15-3 as prognostic factors in breast cancer—univariate and multivariate analysis. Anticancer Res., 19:2545-2550, 1999.

8. Tokunaga, N., Kijima, H., Noto, T., Osamura, Y., Sadahiro, S., Tajima, T., Mitomi, T. Imunohistochemical localization of carcinoembryonic antigen as a predictor of lymph node status in submucosa-invasive colorectal carcinoma. Dis. Colon Rectum., 38:842-847, 1995.

9. Vogel, I., Francksen, H., Soeth, E., Henne-Bruns, D., Kremer, B., and Juhl, H. The carcinoembryonic antigen (CEA) and its prognostic impact on immunocytologically detected intraperitoneal colorectal cancer cells. Am. J. Surg., in press.

10. Gold, P., and Freedman, S. Specific carcinoembryonic antigens of the human digestive system. J. Exp. Med., 122:467-481, 1965.

11. Hammarström, S. The carcinoembryonic antigen (CEA) family:structures, suggested functions and expression in normal and malignant tissues. Semin. Cancer Biol., 2:67-81, 1999.

12. Neumaier, M., Paululat, S., Chan, A., Matthaes, P., and Wagener, C. Biliary glycoprotein, a potential human cell adhesion molecule, is down-regulated in colorectal carcinomas. Proc. Natl. Acad. Sci. USA., 90:10744-10748, 1993.

13. Benchimol, S., Fuks, A., Jothy, S., Beauchemin, N., Shirota, K., Stanners, C. Carcinoembryonic antigen, a human tumor marker, function as an intercellular adhesion molecule. Cell, 57:327-334, 1989.

14. Stanners, C. P., and Fuks, A. 1998. Properties of adhesion mediated by the human CEA families. In: Stanners, C. P. (ed.) Cell adhesion and comunication mediated by the CEA family, pp. 57-71. Amsterdam: Harwood Academic Publishers, 1998.

15. Zhou, H., Fuks, A., Alcaraz, G., Bolling, T. J., and Stanners, C. P. Homophilic adhesion between Ig superfamily carcinoembryonic antigen molecules involves double reciprocal bonds. J. Cell Biol., 122:951-960, 1993.

16. Taheri, M., Saragovi, U., Fuks, A., Makkerh, J., Mort, J., Stanners, C. P. Self recognition in the Ig superfamily. J. Biol. Chem. 275:26935-26943, 2000.

17. Edmiston, K., Gangopadhyay, A., Shoji, Y., Nachman, A., Thomas, P., Jessup, J. M. In vivo induction of murine cytokine production by carcinoembryonic antigen. Cancer Res., 57:4432-4436, 1997.

18. Bayon, L., Izquierdo, M., Sirovich, I., van Rooijen, N., Beelen, R., and Meijer, S. Role of Kupfer cells in arresting circulating tumor cells and controlling metastatic growth in the liver. Hepatology, 23:1224-1231, 1996.

19. Von Kleist, S., Migule, I,, and Halla, B. Possible function of CEA as cell-contact inhibitory molecule. Anticancer Res., 15:1889-1894, 1995.

20. Kammerer, R., and v. Kleist, S. The carcinoembryonic antigen (CEA) modulates effector-target cell interaction by binding to activated lymphocytes. Int. J. Cancer., 68:457-463, 1996.

21. Gangopadhyay, A., Lazure, D., and Thomas, P. Adhesion of colorectal carcinoma cells to the endothelium is mediated by cytokines from CEA stimulated Kupffer cells. Clin. Exp. Metastasis, 16:703-712, 1998.

22. Takahashi, H., Okai, Y., Paxton, R., Hefta, L., Shively, J. Differential regulation of carcinoembryonic antigen and biliary glycoprotein by (−interferon. Cancer Res. 53:1612-1619, 1993.

23. Gossen, M., and Bujard, H. Tight control of gene expression in mammalian cells by tetracycline-responsive promoters. Proc. Natl. Acad. Sci. USA, 89:5547-5551, 1992.

24. Schulte, A., Lai, S., Kurtz, A., Czubayko, F., Riegel, A. T., and Wellstein, A. Human trophoblast and choriocarcinoma expression of the growth factor pleiotrophin attributable to germ-line insertion of an endogenous retrovirus. Proc. Natl. Acad. Sci. USA., 93:14759-14764, 1996.

25. Juhl, H., Downing, S., Wellstein, A., and Czubayko, F. HER-2/neu rate limiting for ovarian cancer growth: conditional depletion of HER-2/neu by ribozyme-targeting. J. Biol. Chem., 272:29482-29486, 1997.

26. Palfner, K., Kneba, M., Hiddemann, W., and Bertram, J. Short technical reports. Quantification of ribozyme-mediated RNA cleavage using silver-stained polyacrylamide gels. Biotechniques, 19 (6):926-91995, 1995.

27. Fang, W. J., Hartmann, N., Chow, D., Riegel, A. T., and Wellstein, A. Pleiotrophin stimulates fibroblasts and endothelial and epithelial cells and is expressed in human cancer. J. Biol. Chem., 267:25889-25897, 1992.

28. Vindelov, L. L., Christensen, I. J., Nissen, N. I. Standardization of high-resolution flow cytometric DNA analysis by the simultaneous use of chicken and trout red blood cells as internal reference standards. Cytometry, 3:323-327, 1983.

29. Freeman, W. E., Robertson, D. J., Vrana, K. E. Fundamentals of DNA hybridization arrays for gene expression analysis. BioTechniques 29:1042-1055, 2000.

30. O'Connell, J., Bennett, M. W., Nally, K., O'Sullivan, G. C., Collins, J. K., Shanahan, F. Interferon-gamma sensitizes colonic epithelial cell lines to physiological and therapeutic inducers of colonocyte apoptosis. J. Cell Physiol., 185(3):331-338, 2000.

31. Nita, M. E., Nagawa, H., Tominaga, O., Tsuno, N., Fujii, S., Sasaki, S., Fu, C. G., Takenoue, T., Tsuruo, T., Muto, T. 5-Fluorouracil induces apoptosis in human colon cancer cell lines with modulation of Bcl-2 family proteins. Br. J. Cancer, 78(8):986-92, 1998.

32. Werner, M., and Uhlenbeck, O. The effect of base mismatches in the substrate recognition helices of hammerhead ribozymes on binding and catalysis. Nucleic Acids Res., 23:2092-2096, 1995.

33. Minami S, Furui J, Kanematsu T: Role of carcinoembryonic antigen in the progression of colon cancer cells that express carbohydrate antigen. Cancer Res 2001; 61(6): 2732-5.

34. Ordonez, C., Screatom, R. A., Ilantzis, C., Stanners, C. P. Human carcinoembryonic antigen functions as a general inhibitor of anoikis. Cancer Res., 60:3419-3424, 2000

33. Minami S, Furui J, Kanematsu T: Role of carcinoembryonic antigen in the progression of colon cancer cells that express carbohydrate antigen. Cancer Res 2001; 61(6): 2732-5.

35. Vogel I, Shen Y, Soeth E, et al.: A human carcinoma model in athymic rats reflecting solid and disseminated colorectal metastases. Langenbecks Arch Surg 1998; 383(6): 466-73.

36. Landuzzi L, Frabetti F, Rossi I, et al.: Expression of transduced carcinoembryonic antigen gene in human rhabdomyosarcoma inhibits metastasis. Cancer Res 1996; 56(19): 4503-8.

37. Thomas P, Gangopadhyay A, Steele G, et al.: The effect of transfection of the CEA gene on the metastatic behavior of the human colorectal cancer cell line MIP-101. Cancer Lett 1995; 92(1): 59-66.

38. Bajenova O V, Zimmer R, Eugenia S, Salisbury-Rowswell J, Nanji A, Thomas P: HnRNP M4 is a receptor for carcinoembryonic antigen in Kupffer cells. J Biol Chem 2001; 13:13.

39. Engler S, Thiel C, Forster K, David K, Bredehorst R, Juhl H: A novel metastatic animal model reflecting the clinical appearance of human neuroblastoma: growth arrest of orthotopic tumors by natural, cytotoxic human immunoglobulin M antibodies. Cancer Res 2001; 61(7): 2968-73.

What is claimed is:

1. A method of treatment or prophylaxis of cancer comprising administering to a subject in need thereof an effective amount of an agent capable of inhibiting the expression of carcinoembryonic antigen (CEA).

2. The method of claim 1, wherein the agent is a ribozyme.

3. The method of claim 2, wherein the ribozyme is expressed by an oligonucleotide comprising a nucleic acid sequence selected from the group consisting of TGCTCTT; ACTATGGA; TCCATAGT; AAGAGCA; CTGATGAGTCCGTTAGGACGAA; TTCGTCCTAA CGGA CTCA TCAG; TGCTCTTCTGATGAGTCCGTTAGGACGAAACTATGGA; TCCATAGTTTCGTCCTAACGGACTCATCAGAAGAGCA; 5′-agcttTGCTCTTCTGATGAGTCCGTTAGGACGAAACTATGGAgggcc-3′; and 5′ -cTCCATAGTTTCGTCCTAACGGACTCATCAGAAGAGCAa-3′.

4. The method of claim 2, wherein the ribozyme is expressed by an oligonucleotid comprising 5′xTCCATAGTTTCGTCCTAACGGACTCATCAGAAGAGCAy-3′; wherein x and y are independently one or more of any of A, C, G and T.

5. The method of claim 2, wherein the ribozyme is cable of cleaving CEA mRNA.

6. The method of claim 1, wherein the cancer is selected from the group consisting of colon; breast; lung; cervical; prostate; and head and neck cancer.

7. The method of claim 1, wherein the cancer is colon cancer.

8. A method of potentiating or enhancing the effect of a cancer treatment comprising in addition to said cancer treatment administering to a subject in need thereof an effective amount of an agent capable of inhibiting the expression of CEA.

9. The method of claim 8, wherein the agent is a ribozyrne.

10. The method of claim 9, wherein the ribozyme is expressed by an oligonucleotide comprising a nucleic acid sequence selected from the group consisting of TGCTCTT; ACTATGGA; TCCATAGT; AAGAGCA; CTGATGAGTCCGTTAGGACGAA; TTCGTCCTAACGGACTCATCAG; TGCTCTTCTGATGAGTCCGTTAGGACGAAACTATGGA; TCCATAGTTTCGTCCTAACGGACTCATCAGAAGAGCA; 5′-agcttTGCTCTTCTGATGAGTCCGTTAGGACGAAACTATGGAgggcc-3′; and 5′-cTCCATAGTTTCGTCCTAACGGACTCATCAGAAGAGCAa-3′.

11. The method of claim 9, wherein the ribozyme is expressed by an oligonucleotid comprising 5′xTCCATAGTTTCGTCCTAACGGACTCATCAGAAGAGCAy-3′; wherein x and y are independently one or more of any of A, C, G and T.

12. The method of claim 9, wherein the ribozyme is cable of cleaving CEA mRNA.

13. The method of claim 8, wherein the cancer is selected from the group consisting of colon; breast; lung; cervical; prostate; and head and neck cancer.

14. The method of claim 8, wherein the treatment comprises chemotherapy, radiation, and/or antisense therapy.

15. A ribozyme expressed by an oligonucleotide comprising a nucleic acid sequence selected from the group consisting of TGCTCTT; ACTATGGA; TCCATAGT; AAGAGCA; CTGATGAGTCCGTTAGGACGAA; TTCGTCCTAACGGACTCATCAG; TGCTCTTCTGATGAGTCCGTTAGGACGAAACTATGGA; TCCATAGTTTCGTCCTAACGGACTCATCAGAAGAGCA; 5′-agcttTGCTCTTCTGATGAGTCCGTTAGGACGAAACTATGGAgggcc-3′; and 5′ -cTCCATAGTTTCGTCCTAACGGACTCATCAGAAGAGCAa-3′.

16. The ribozyme of claim 15, wherein the ribozyme is expressed by an oligonucleotid comprising 5′xTCCATAGTTTCGTCCTAACGGACTCATCAGAAGAGCAy-3′; wherein x and y are independently one or more of any of A, C, G and T.

17. An isolated antibody that binds specifically to the CEA polypeptide.

18. A monoclonal antibody according to claim 17.

19. A method of inhibiting apoptosis or proliferation of a cancer cell, comprising inhibiting expression of CEA in said mammalian cell.

20. The method of claim 19, wherein said mammalian cell is transformed with a vector encoding an antisense oligonucleotide corresponding tot he CEA sequence.

21. An antisense oligonucleotide that inhibits the expression of CEA in a mammalian cell and has a phosphodiester backbone or modified base composition.

22. The antisense oligonucleotide of claim 22 which is contained in a liposomal formulation.

23. A method of treating cancer characterized by CEA overexpression by administration of an antisense oligonucleotide, ribozyme or small interfering RNA (SI RNA) molecule that inhibits CEA expression.

24. A method of treating cancer characterized by CEA overexpression comprising administering an antibody that specifically binds to CEA.

25. A method of treating cancer characterized by CEA overexpression comprising administration of an antibody that specifically binds to CEA, antisense oligonucleotide, ribozyme or small interfering RNA (SI RNA) molecule in combitation with radiation, radionucleides, anticancer drugs or other biological agents.

26. A method of treating cancer characterized by CEA overexpression comprising administration of antibody that specifically binds CEA, antisense oligonucleotide, ribozyme or small interfering RNA (SI RNA) molecule contained in a liposomal formulation, in combination with radiation, radionucleides, anticancer drugs or other biological agents.

27. A method for identifying small molecule inhibitors of the CEA protein, wherein the method comprises the steps of:
(a) determining a three dimensional structure of the CEA protein;
(b) identifying an active site in the structure determined in step (a);
(c) computationally screening a database of compounds to identify molecules that fit in the active site of the protein and selecting the molecules with the highest calculated binding affinity to the protein; and
(d) testing in vitro the CEA inhbitory activity of the molecules selected in step (c) and identifying one or more CEA inhibitors.

28. The method of claim 27, wherein determining the three dimensional structure of the CEA protein comprises determining the structure through X-ray crystallography.

29. The method of claim 27, wherein determining the three dimensional structure of the CEA protein comprises identifying a protein of known structure that is homologous to CEA and modeling the structure of the CEA protein based on the structure of the homologous protein.

30. A method for inhibiting cancer cell proliferation and/or metastasis in a cancer patient comprising administering to the patient a therapeutically effective amount of a compound identified according to claim 27.

31. A method for designing small molecule inhibitors of the CEA protein represented by the polypeptide of FIG. 1, wherein the method comprises the steps of:
(a) determining a three dimensional structure of the CEA protein;
(b) identifying an active site in the structure determined in step (a);
(c) computationally modeling a compound that is complementary to the active site of the CEA protein; and
(d) testing in vitro the CEA inhbitory activity of the molecules selected in step (c) and identifying one or more CEA inhibitors.

33. The method of claim 31, wherein determining the three dimensional structure of the CEA protein comprises determining the structure through X-ray crystallography.

34. The method of claim 31, wherein determining the three dimensional structure of the CEA protein comprises identifying a protein of known structure that is homologous to CEA and modeling the structure of the CEA protein based on the structure of the homologous protein.

35. A method for inhibiting cancer cell proliferation and/or metastasis in a cancer patient comprising administering to the patient a therapeutically effective amount of a CEA inhibitor designed according to claim 31.
